(19) United States
(12) Patent Application Publication
(10) Pub. No.: US 2005/0169840 A1
(43) Pub. Date: Aug. 4, 2005

(54) Method for screening modulators of the immune system

(76) Inventor: Jacob Hochman, Jerusalem (IL)

Correspondence Address: NATH & ASSOCIATES, 1030 15th STREET, NW, 6TH FLOOR, WASHINGTON, DC, 20005, US

(21) Appl. No.: 10/519,090

(22) PCT Filed: Jun. 25, 2003
(86) PCT No.: PCT/IL03/00536

Related U.S. Application Data
(60) Provisional application No. 60/390,886, filed on Jun. 25, 2002.

(51) Int. Cl. A61K 49/00; A01K 67/027

(52) U.S. Cl. 424/9.2; 800/18

(57) ABSTRACT

A method for determining whether a factor is a modulator of the transmission, of immunity from a female mammal to newborn mammals by consumption of milk from the female. A female mammal, such as a BALB/C female mouse, is exposed to the factor, and then immunized to one or more antigens. The female is then made to lactate, and newborn mammals are allowed to consume milk obtained from the lactating female. The newborns are then inoculated with the one or more antigens. The level of immunity in the newborns to the one or more antigens is then compared to that in control newborns that received milk from a female that was immunized against the one or more antigens but not exposed to the factor. A difference between the level of immunity to the one or more antigens in the experimental newborns and the control newborns is indicative that the factor is a modulator of the transmission of immunity to the one or more antigens from a female mammal to newborn mammals by consumption of milk from the female.

### BACKGROUND OF TIE INVENTION

Ocular lymphoma is a lethal disease caused mainly by two clinically distinct forms of non-Hodgkin's lymphoma (1) non-Hodgkin's lymphoma of the central nervous system (NHL-CNS) and (2) systemic lymphoma metastatic to the eye. Hodgkin's lymphoma very rarely causes ocular and orbital diseases. The NHL-CNS form arises within the brain, spinal cord, leptomeninges or the eye, but then may spread throughout the CNS, with rare systemic spread outside the CNS. In contrast, systemic non-Hodgkin's lymphoma almost always arises outside of the CNS. The disease is aggressive, and most patients die within 1 to 5 years of diagnosis.

T-25-Adh is a cell line that is non-tumorigenic and immunogenic in mature Balb/C mice. This cell line was derived from tumorigenic, suspension borne T-25 (lymphoma) cells through selection for spontaneous substrate-adhering cell variants^{(5,6)}. Rev-2-T-6 is a revertant tumorigenic cell line (ECACC Accession No. 94122103) derived from T25-Adh cells^{(5,6)}. Thus, a full cycle from tumorigenic (T-25 cells), to immunogenic, non-tumorigenic (T-25-Adh cells), and back to the tumorigenic state (Rev-2-T-6 cells) has been achieved. Postnatal and mature mice inoculated with Rev-2-T-6 cells develop systemic lymphoma. After intraperitoneal (i.p.) inoculation of Rev-2-T-6 cells into newborn mice (optimum: 7 days postnatal), specific infiltration of these cells to the brain and eyes occurs in 60% of inoculated mice⁽¹⁻²⁾. Infiltration is not observed in mice inoculated later than day 11 postnatal. The infiltration of Rev-2-T-6 cells to the brain and eyes is first observed through clinical signs of eye and CNS involvement, including unilateral or bilateral involvement of the orbit and eyelids, accumulation of lymphoma cells in the anterior chamber of the eye (masking the posterior surface of the cornea), retardation of animal growth, ataxia, spinning when held by the tail, and arched backs. Subsequent histological analysis reveals tumorous infiltration into a variety of structures of the orbit, intraocular tissues, along the optic nerve, and in the brain⁽²⁾.

The Rev-2-T-6 cells enter the brain, preferentially, through the choroid plexus, cranial nerves and cranial nerve ganglia⁽³⁾. Infiltration of the rostral part occurs prior to the caudal part of the brain. Once within the brain, the cells spread within it as well as migrate along the optic nerve sheath into the eyes, where they continue to migrate along the choroid, ciliary body, iris and into the anterior chamber of the eye. The orbit is also infiltrated by the Rev-2-T-6 cells. However, this metastasis is carried out independent of the brain optic nerve-intraocular route⁽³⁾.

When Balb/C females were immunized with T-25-Adh cells, impregnated and their offspring inoculated at day 7 postnatal with Rev-2-T-6 cells, about 70% of the offspring (N=33) survived over 120 days post inoculation. In a control group in which the mothers were not immunized with T-25-Adh cells 20% of the offspring (N=39)survived over 120 days, with a median survival of 58 days. Abdominal, orbital and CNS manifestations of lymphoma were also reduced in the experimental group when compared with the control group. When newborn mice were exchanged between immunized and non-immunized mothers, those newborns born to a non-immunized mother and nursed by an immunized female acquired immunity against a subsequent challenge with Rev-2-T-6 cells. Milk from the immunized females contains antibodies that recognize Rev-2-T-6 cells by western blot analysis⁽⁴⁾.

### DETAILED DESCRIPTION OF THE INVENTION

The present invention provides a method for screening and identifying factors having an effect on the transmission of immunization of newborns through consumption of milk from immunized females. In accordance with the invention, a female mammal is subjected to a factor and then immunized against one or more antigens. The female is subsequently caused to lactate and newborn mammals are allowed to consume milk from the female. The female may be caused to lactate by being impregnated and the newborn mammals may be female's own offspring. The offspring are then tested for immunity against the antigen or antigens. If the newborns acquire a level of immunity against the antigens that is significantly different (higher or lower) than that observed in control newborns (who consumed milk from a female that was not immunized against the antigens), the factor is a modulator (enhancer or suppresser, respectively) of the transmission of immunization of newborns acquired through consumption of milk.

The term “modulator” as used herein should be construed in the broad sense and includes any factor having a positive or negative effect on the transmission of immunization (i.e. a factor enhancing or suppressing the immune system, respectively) from an immunized female to a newborn, e.g. from a mother to her offspring.

The factor may be an environmental factor, including exposure to a chemical compound or a mixture of chemical compounds, biological macromolecules, exposure to air pollutants, smoke (direct or indirect) or smoke extracts, exposure to different levels of oxygen, irradiation of various types (e.g. UV, radioactive, radiations caused by cellular phones or communication antennas); the factor may be a chemical or biological agent (drugs, pharmaceuticals, agricultural agents e.g. insecticides, pesticides; toxin, lead, agents of chemical warfare, food additives); the factor may also be a nutritional factor including providing the lactating female with elevated (or reduced) levels of vitamins and minerals, a natural product such as an extract made from biological materials or any other food additive or food supplemental; the factor may also be a psychological factor including stress causing or relaxing factors etc. The factor may also be a drug (used, for example, in chemotherapy or immune therapy, or for that matter any drug that can be found to affect the transfer of immunity from mother to offspring).

Also, the effect of drug interactions on transfer of immunity from a lactating female to a newborn might also be taken into account.

The factor may also be a psychological factor that is applied by the use of psychological evaluating models known to those versed in the art of psychology, including the labyrinth model or any other model for applying the tested modulator onto the animal.

The manner by which the female is exposed to the modulator depends, a priori on the type of modulator and may be determined easily by the practitioner. For example, exposure to gaseous material (chemicals, pollutants, smoke, oxygen) may be performed by placing the animal in a special chamber (e.g. a smoking chamber) containing means for exposing the animal to predefined levels of the gas and at a predefined schedule. Liquid, semi-liquid or solid substances may be formulated for administration, e.g. by topical application, oral administration (e.g. as a food additive, in the drinking water), by injection etc.

The term “inoculation” as used herein should be construed as any means of systemic administration of a substance to a subject. That is, a means of administration that is not directed into a specific target organ, and includes oral, subcutaneous or parenteral administration, including intravenous (i.v.), intraarterial (i.a.), intramuscular (i.m.), intraperitoneally (i.p.) and intranasal (i.n.) administration as well as intrathecal and infusion techniques.

The term “disease” according to the invention should be construed as any disease against which a humoral (immunity mediated by antibodies) and/or a cellular immune response may be produced. The disease may be cancer, an infection with a pathogen (bacterial, fungal, viral etc.), an allergy etc.

“Causing said immunized female animal to lactate” as used herein above and below should be construed in its broad sense as referring to any condition, which yield a lactating female animal (i.e. a female animal which produces breast-milk). Such conditions include impregnation of the female animal or providing the animal with a suitable hormonal treatment.

The female animal, which may be used herein interchangebly with the term “animal host”, may be any immunocompetent host and can be selected from a range of mammals including those animals customarily used in laboratories. A specific example includes Balb/C mice. Alternatively, the animal host may be an immunodeficient host, e.g., a genetically immunodeficient rodent such as the SCID, Nude, Beige and BNX mice, or a rodent which was immunocompromised by irradiation or appropriate chemical treatment and whose immune system was then reconstituted by a bone marrow of SCID mice, such as that described in European Patent Applications Nos. 438053 and 517199. The newborn is preferably of the same species as the lactating animal.

According to yet another aspect of the invention there is provided a method for modulating immunity towards a disease transferred from an immunized female animal to a newborn, the method comprising exposing said immunized female animal to a modulator or combination of modulators for a time sufficient to induce a therapeutic effect, causing said immunized female animal to lactate and feeding said newborn with milk derived from the lactating female animal; the therapeutic effect being modulation (preferably, enhancement) of the immunity against said disease transferred to the newborn.

One preferred embodiment of this aspect of the invention is the identification

According to one embodiment of the invention, the disease towards which immunity is conferred is cancer, preferably, lymphoma and, more preferably, non-Hodgkin's lymphoma.

The newborns are inoculated with a tumorigenic cell line. In case the tumor cells are lymphoma cells, and in particular, Rev-2-T-6 cells, it has been shown that the optimum conditions for effective homing of tumorigenic cells into target tissues (e.g. the eyes and brain) of non-immunized newborns is 7 days postnatal. Thus, according to one preferred embodiment, the immunized newborns are inoculated at days 1 to 11 postnatal. Inoculation may take place either during or after termination of suckling from the lactating animal.

As indicated above, the newborns are preferably fed with milk from an immunized female host and more preferably the newborns are offspring of immunized mouse. Inoculation of the newborns may take place either during or after a period of receiving milk from the immunized female (e.g. during of after termination of sucking).

The invention also concerns a method for modulating immunity towards a disease transferred from an immunized female animal to a newborns the method comprising exposing the immunized female animal to a modulator or combination of modulators for a time sufficient to induce an effect, preferably, an effect having a therapeutic benefit, causing the immunized female animal to lactate and feeding the newborn with mild derived from the lactating animal; the effect being modulation (preferably enhancement or stimulation) of immunity against said disease transferred from the immunized animal to the newborn.

The disease against which immunity is conferred is preferably cancer, and more preferably lymphoma. A specific type of lymphoma is non-Hodgkin's lymphoma. The modulator may be any factor derived from environmental, nutritional, physiological, psychological origin or any other factor as defined above.

According to, one embodiment, the method may be useful for sensitizing multi-drug resistant (MDR) cancer cells to chemotherapy. This embodiment is referred to herein as the MDR-reversing embodiment. It should be noted that this embodiment of the invention provides a non-invasive, in vivo assay for investigating putative NMR-reversing agents, which is the closest to the conditions provided in in vitro systems, i.e. in culture.

The ability of tumor cells to develop multiple drug resistance (OR) is frequently observed in cell culture systems and is a major factor limiting the clinical success of many currently used anti-cancer drugs. Lymphoma cells may become drug resistant either spontaneously, or in an in vitro culture, by transformation with a MDR gene or by selection, for example in increasing concentrations of colchicine.

One prominent mechanism of MDR is overexpression of membrane transport proteins, which effectively remove anti-cancer drugs from the tumor cells. The prototypical transporter is a 170 kD transmembrane protein, P-glycoprotein, that mediates the efflux of many structurally unrelated natural products including anti-cancer drugs, such as anthracyclines, vinca alkaloids, Taxol, epipodophyllotoxins, and certain antibiotics. Enhanced efflux of these compounds reduces their intracellular accumulation and so reduces their cytotoxicity. Another drug transporter called MRP has been identified and may be involved in resistance to anti-cancer drugs.

Several types of compounds that circumvent P-glycoprotein-mediated MDR have been isolated and are being characterized. These agents fall into two categories based on their interactions with P-glycoprotein, i.e., agents that potentiate the cytotoxicity of classical anti-cancer drugs by inhibiting the function of P-glycoprotein, or agents that act as direct cytotoxins to both drug-sensitive and MDR cells.

The invention may therefore be used to identify agents, which circumvent MDR. Such compounds are also referred to as MDR-reversing agents.

According to this aspect of the invention, a healthy animal is first inoculated with MDR cancer cells. The MDR cancer cells may be obtained by in vitro selection in increasing concentrations of colchicine or by transformation of non-resistant cells with MDR gene (for example, from human or murine source).

The marker infiltrates the malignant cells only upon reversing the resistance of the cells, i.e. after reducing or preventing the efflux mediated by P-glycoprotein. The marker may be any agent, which produces a signal after circumventing resistance. One example of such a marker is a fluorescent tag (dye) which penetrates: the cells upon said circumvention, thereby labeling the cells with a fluorescent dye. The marker as well as the screened agents are provided to the animal host by any conventional administration route, e.g. by oral administration, injection, etc., however, according to a preferred embodiment, are provided topically, for example, as eye drops.

According to a preferred embodiment of this aspect of the invention, the animal is a rodent, and more particularly, Balb/C mice. In this specific case, the healthy mice may be inoculated with the tumorigenic T-25-Adh lymphoma cells transformed with a MDR gene (or obtained by selection). In the following specific example, the T-25-Adh lymphoma cells are obtained by transformation of MDR gene from human source.

Thus, in its first aspect, the invention provides a method for determining whether a factor is a modulator of the transmission of immunity to one or more antigens from a female mammal to newborn mammals by consumption of milk from the female, the method comprising:
(a) immunizing a first female mammal against the one or more antigens.
(b) exposing the first female mammal to the factor;
(c) causing the first female mammal to lactate;
(d) allowing a first group of one or more newborn mammals to consume milk obtained from the first lactating female;
(e) inoculating the first group of newborns with the one or more antigens; and
(f) comparing a level of immunity to the one or more antigens in the first group of newborns with a level of immunity to the one or more antigens in a second group of newborns that consumed milk from a second lactating female that was immunized against the one or more antigens but not exposed to the factor, a difference between the level of immunity to the one or more antigens in the first group of newborns and the level of immunity to the one or more antigens in the second group of newborns being indicative that the factor is a modulator of the transmission of immunity to the one or more antigens from a female mammal to newborn mammals by consumption of milk from the female.

In a second aspect, the invention provides a method for determining whether a factor is a modulator of the transmission of immunity to non-Hodgkin's lymphoma from a from a female Balb/C mouse to newborn mammals by consumption of milk from the female Balb/C mouse, the method comprising:
(a) inoculating a first female mouse with T-25-Adh cells so as to immunize the first female mouse to the non-Hodgkin's lymphoma;
(b) exposing the first female mouse to the factor;
(c) causing the first female mouse to lactate;
(d) allowing a first group of one or more newborn Balb/C mice to consume milk obtained from the first lactating mouse;
(e) inoculating the first group of newborn mice with a tumorigenic Rev-2-T-6 cell line which is capable of infiltrating into the eye, the central nervous system (CNS) of the first group of newborn, or to develop to systemic lymphoma; and
(f) comparing a level of immunity to the in the first group of newborns with a level of immunity to the non-Hodgkin's lymphoma in a second group of newborns that consumed milk from a second lactating female Balb/C mouse that was immunized against the non-Hodgkin's lymphoma but not exposed to the factor, a difference between the level of immunity to the non-Hodgkin's lymphoma in the first group o f newborns and the level of immunity to the non-Hodgkin's lymphoma in the second group of newborns being indicative that the factor is a modulator of the transmission of immunity to the non-Hodgkin's lymphoma from a female Balb/C mouse to newborn Balb/C mice by consumption of milk from the female.

In another of its aspects, the invention provides a method for modulating transmission of immunity to one or more antigens from a female mammal to newborn mammals by consumption of milk from the female, the method comprising exposing a female animal immunized to the one or more antigens to a modulator of the transmission of immunity to the one or more antigens from a female mammal to newborn mammals by consumption of milk for a time sufficient to induce modulation of the transmission of immunity against the antigens by consumption of milk, causing the immunized female mammal to lactate and allowing newborn mammals to consume milk from the lactating female mammal.

A method of identifying agents which sensitize lymphoma cells against a drug, the method comprising:
(a) providing an animal host having lymphoma which infiltrated the eye of the animal;
(b) providing said animal host with a marker which infiltrates the lymphoma cells only upon sensitization of said cells to a chemotherapeutic drug;
(c) providing said animal host with said agent; and
(d) determining infiltration of said marker into said lymphoma cells, said infiltration indicting the sensitization of said cancer cells to chemotherapy by said agent.

### EXAMPLES

Materials and Methods

Cells

T-25-Adh cells were obtained from tumorigenic, suspension borne T-25 cells through spontaneous selection for substrate adhering variants^{(5,6)}.

Rev-2-T-6 cells were derived from substrate-adherent, nontumorigenic (immunogenic) variants of the S49 mouse T-cell lymphoma, the T-25-Adh, as described previously⁽²⁾. In general, cell-substrate adherent T-25-Adh cells were propagated in Dulbecco's Modified Eagle's Medium (DMEM) (Gibco) supplemented with 10% heat inactivated horse serum (Biological Industries—Beth Haemek, Israel), Penicillin (50 u/mil) and Streptomycin (50 mg/ml), Cells were maintained at 37° C. in a humidified atmosphere containing 5% CO₂. The adherent cells were selected in vitro for Ouabain resistance (OUA-R). The resulting T-25-Adh cells were subjected in vitro to 30-50 mJoule of TV radiation. Culture plates in which 10-20% of the cells survived following the irradiation were further propagated and selected for cells growing in suspension. The resulting “revertant” cells, termed “Rev-2” cells were then injected into Balb/C mice at a dosage of 10⁷ cells/mouse and, after six passages in mice, the tumorigenic revertants, termed “Rev-2-T-6” cells were obtained. These cells grow in vitro in the form of a suspension of large clumps.

Animals

Syngeneic Balb/C mice were obtained from the animal facility of the Hebrew University of Jerusalem.

### Example 1

Balb/C females are immunized with T-25-Adh cells. The females are exposed at a predefined time schedule and predefined dosages, to cigarette smoke using a smoke machine with a direct exposure equivalent to 10 cigarettes/day. The females are impregnated either after or during the exposure to the smoke. One week after starting exposure to smoke, female mice are inoculated intraperitoneally with 10⁷ T-25-Adh cells. Four weeks later, a booster of 10⁷ T-25-Adh cells is given intraperitoneally. Two weeks after booster inoculation, the females are impregnated. Exposure to smoke is continued throughout the above mentioned time period until the day of birth. On day 7 postnatal, the offspring are inoculated intraperitoneally with 5×10⁶ Rev-2-T-6 cells and followed thereafter for signs of systemic, CNS and ocular lymphoma. As a continuation of the experiment, mice that survive the inoculation of Rev-2-T-6 cells are inoculated at 8 weeks postnatal with T-25 cells^{(6,7)} and are followed thereafter for survival and signs of systemic lymphoma development. As a reference, (control), the same experiment is carried out on female mice (and their offspring) that are not exposed to smoke. As a further reference (second control), the same experiment is carried out on non-immunized mothers (and their offspring).

The offspring are then inoculated at day 7 postnatal with Rev-2-T-6 cells and the level of transmission of anti-lymphoma immunity from the mother to the offspring as a result of exposure to the smoke is determined. The level of immunity is compared to that of the control groups. A difference in the level of immunity in the experimental offspring (whose mother was exposed to smoke) in comparison to that of the control offspring(whose mother was not exposed to smoke) indicates that cigarette smoke is a modulator of the transmission of immunity from a female to offspring by consumption of milk

### Example 2

Non-tumorigenic, immunogenic T-25-Adh cells were transduced with a retrovirus containing the human MDR1 cDNA and inoculated into BALB/C mice⁽⁸⁾. The resultant multi-drug resistant cells (named HU-1) demonstrated an enhanced (45%-50% of mice) infiltration to the eye, when compared to T-25-adh cells⁽⁵⁾.

The above MDR mice are used for screening MDR reversing agents, i.e. agents which sensitize lymphoma cells against a drug. In particular, the MDR animals are provided with (i) drops of fluorescent dyes which are applied directly to the eyes of the mice which are infiltrated by the MDR lymphoma cells, followed by (ii) drops of the potential reversing agent which are applied directly to the eyes of the mice either concomitant or after administration of the marker. After a time period, the infiltration of the marker to the lymphoma cells is determined, the infiltration being indicative of the sensitization of said cancer cells to chemotherapy by the potential agent. For example, application of an inactive drug leaves the lymphoma cells in the anterior chamber devoid of fluorescence.

As a control group animals inoculated with sensitive lymphoma cells are used.

The infiltration (or none) of the dye to the lymphoma cells is visualized through a non-invasive fluorescence optics.

Once the reversing agent is identified, it is inoculated systemically to be followed by administration of a chemotherapeutic drug (to which the lymphoma cells are resistant prior to their inoculation into mice) to determine whether the lymphoma cells have been sensitized thereto.

1-43. (canceled)

44. A method for determining whether a factor is a modulator of the transmission of immunity to one or more antigens from a female mammal to newborn mammals by consumption of milk from the female, the method comprising:
(a) immunizing a first female mammal against the one or more antigens.
(b) exposing the first female mammal to the factor;
(c) causing the first female mammal to lactate;
(d) allowing a first group of one or more newborn mammals to consume milk obtained from the first lactating female;
(e) inoculating the first group of newborns with the one or more antigens; and
(f) comparing a level of immunity to the one or more antigens in the first group of newborns with a level of immunity to the one or more antigens in a second group of newborns that consumed milk from a second lactating female that was immunized against the one or more antigens but not exposed to the factor, a difference between the level of immunity to the one or more antigens in the first group of newborns and the level of immunity to the one or more antigens in the second group of newborns
(g) being indicative that the factor is a modulator of the transmission of immunity to the one or more antigens from a female mammal to newborn mammals by consumption of milk from the female.

45. The method of claim 44 wherein the one or more antigens causes a disease.

46. The method of claim 45 wherein the disease is cancer.

47. The method of claim 46, wherein the cancer is non-Hodgkin's lymphoma.

48. The method of claim 44 wherein immunizing the first female mammal against the one or more antigens involves inoculating the first female with T-25-Adh cells.

49. The method of claim 48, wherein comparing a level of immunity to the one or more antigens in the first group of newborns with a level of immunity to the one or more antigens in a second group of newborns is performed during or after termination of consumption of the milk obtained from the first or second lactating female.

50. The method of claim 44, wherein the factor is selected from the group comprising:
(a) an environmental factor;
(b) a chemical compound or a mixture of chemical compounds;
(c) a biological macromolecule;
(d) air pollutants
(e) direct or indirect cigarette smoke or smoke extracts;
(f) extreme oxygen pressures;
(g) ultraviolet or radioactive irradiation;
(h) radiation produced by cellular phones or communication antennas;
(i) a drug or pharmaceutical;
(j) an agricultural agent;
(k) an insecticides or pesticide
(l) a toxin;
(m) lead
(n) an agent of chemical warfare;
(o) a food additives;
(p) a nutritional factor
(q) a vitamin or dietary mineral;
(r) a psychological factor
(s) a stress causing or relaxing factor.

51. The method of claim 49, wherein comparing a level of immunity to the one or more antigens in the first group of newborns with a level of immunity to the one or more antigens in a second group of newborns involves inoculation the first and second group of newborns with a tumorigenic cell line.

52. The method of claim 51, wherein said tumorigenic cell line comprises Rev-2-T-6 cells.

53. A method for determining whether a factor is a modulator of the transmission of immunity to non-Hodgkin's lymphoma from a from a female Balb/C mouse to newborn mammals by consumption of milk from the female Balb/C mouse, the method comprising:
(a) inoculating a first female mouse with T-25-Adh cells so as to immunize the first female mouse to the non-Hodgkin's lymphoma;
(b) exposing the first female mouse to the factor;
(c) causing the first female mouse to lactate;
(d) allowing a first group of one or more newborn Balb/C mice to consume milk obtained from the first lactating mouse;
(e) inoculating the first group of newborn mice with a tumorigenic Rev-2-T-6 cell line which is capable of infiltrating into the eye, the central nervous system (CNS) of the first group of newborn, or to develop to systemic lymphoma; and
(f) comparing a level of immunity to the in the first group of newborns with a level of immunity to the non-Hodgkin's lymphoma in a second group of newborns that consumed milk from a second lactating female Balb/C mouse that was immunized against the non-Hodgkin's lymphoma but not exposed to the factor, a difference between the level of immunity to the non-Hodgkin's lymphoma in the first group of newborns and the level of immunity to the non-Hodgkin's lymphoma in the second group of newborns being indicative that the factor is a modulator of the transmission of immunity to the non-Hodgkin's lymphoma from a female Balb/C mouse to newborn Balb/C mice by consumption of milk from the female.

54. The method of claim 53, wherein said factor is selected from the group comprising:
(a) an environmental factor;
(b) a chemical compound or a mixture of chemical compounds;
(c) a biological macromolecule;
(d) air pollutants
(e) direct or indirect cigarette smoke or smoke extracts;
(f) extreme oxygen pressures;
(g) ultraviolet or radioactive irradiation;
(h) radiation produced by cellular phones or communication antennas;
(i) a drug or pharmaceutical;
(j) an agricultural agent;
(k) an insecticides or pesticide
(l) a toxin;
(m) lead
(n) an agent of chemical warfare;
(o) a food additives;
(p) a nutritional factor
(q) a vitamin or dietary mineral;
(r) a psychological factor
(s) a stress causing or relaxing factor.
